(19) United States
(12) Patent Application Publication
(10) Pub. No.: US 2007/0254853 A1
(43) Pub. Date: Nov. 1, 2007

(54) Compositions and Methods for Promotion of Wound Healing

(75) Inventors: Charles Van Buren, Houston, TX (US); Anil Kulkarni, Pearland, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 11/741,730

(22) Filed: Apr. 28, 2007

Related U.S. Application Data
(60) Provisional application No. 60/796,489, filed on May 1, 2006.

(51) Int. Cl. A61K 48/00; A61K 31/7076; A61K 31/7072

(52) U.S. Cl. 514/44; 514/46; 514/49

(57) ABSTRACT

The present invention includes compositions and methods for the treatment of animals that are immunosuppressed by preparing a topical composition supplemented with one or more compounds selected from the group consisting of nucleotides, nucleosides, nucleobases and mixtures thereof in an amount effective for promoting wound healing and contacting the wound of the immunosuppressed mammal with the topical composition.

### CROSS-REFERENCE TO RELATED APPLICATIONS

This application claims priority to U.S. Provisional Patent Application Ser. No. 60/796,489, filed May 1, 2006, the entire contents of which are incorporated herein by reference.

### TECHNICAL FIELD OF THE INVENTION

The present invention relates generally to methods and compositions for promotion of wound treatment, and more particularly, to compositions of matter and methods that promote wound healing in animals that are immunosuppressed.

### BACKGROUND OF THE INVENTION

Without limiting the scope of the invention, its background is described in connection with wound healing.

The usefulness of nucleotides in the repair or regeneration of intestinal gut cells in infants was the basis of the U.S. Pat. No. 4,994,442, issued to Gil, et al. This patent relates to methods for stimulation or repair and regeneration of intestinal gut cells in infants and enhancing the immune response of T-cells. Briefly, Gil, et al., teach a milk and non-milk based infant formula that includes nucleosides and/or nucleotides. The process of intestinal repair is continual and physiologically distinct from wound healing in response to trauma or insult.

A role for dietary nucleotides in preventing the onset of infection has also been described in U.S. Pat. No. 4,486,439, issued to Studt et al. These inventors teach a method for the preventive and curative treatment of coccidial infections in humans and other animals by the administration thereto of an amidinourea of amidinothiourea. Briefly, Studt, et al., disclose a method for treating coccidial infections employing a formulation that includes, among other ingredients, 2-pyrimidine, 4-pyrimidine, 5-pyrimidine, 6-pyrimidine, 2-purine, 6-purine, 8-purine or 9-purine.

Dietary nucleotides have been implicated as having a role in relation to delayed cutaneous hypersensitivity (Kulkami, et al. 1987) and in the fatty acid composition of erythrocyte membrane lipids in infants (DeLucchi, et al. 1987).

Another composition is taught in U.S. Pat. No. 5,066,500, also issued to Gil et al., for infant formulas and nutrition products enriched with nucleosides and/or nucleotides and processes for their preparation.” Briefly, Gil, et al., teach that nucleosides and/or nucleotides are added to non-milk based infant formulas to provide a formula having enhanced physiological properties and also closely resemble human milk. Additionally, nutritionally balanced diet formulations are described having nucleosides and/or nucleotides incorporated therein.

A respiratory enzyme booster tablet that includes a combination of diphosphopyridine nucleotide, nicotinamide, adenosine-5-monophosphate and a carrier has been described in U.S. Pat. No. 4,308,257, issued to Caspe. Caspe teaches a composition of matter and method for introducing the composition of matter into the human body to achieve cellular repair. The composition has as a first component a stable, pyrogen-free liquid mixture containing an amino acid metabolite, a thiamine salt, diphosphopyridine nucleotide, diaphorase flavin protein enzyme, and a carrier in select ratios, and as a second component an enteric coated tablet comprising nicotineamide, adenosine-5-monophosphate, diphosphopyridine nucleotide, and an inert carrier in select ratios. The first component is administered by subcutaneous injection, followed by ingestion of the second component. The invention is said to be particularly useful in healing ulcers, burns, post-operative wounds, and various skin disruptions of diabetics.

### SUMMARY OF THE INVENTION

The problems associated with wound healing during immunosuppressive therapy are remedied by the compositions and methods of the present invention. The inventors have found that wound healing of immunosuppressed mammals can be greatly enhanced by the inclusion of nucleotides and/or substances that include essential nucleotides, such as RNA, DNA, oligonucleotides, purine and pyrimidine bases, or any other source in a topical pharmaceutical preparation. The nucleotides are provided in nutritionally effective amounts at a wound site, e.g., to enhance wound healing of patients that are undergoing immunosuppressive therapy. Examples of patients undergoing immunosuppressive therapy include patients that have received transplants and are undergoing a regime of therapy to reduce or eliminate transplant rejection. Transplant surgery will benefit from the present invention as the locations of the surgical wound may be treated before, during or after the surgery. In fact, the present invention may be used for enhancing wound healing of patients with autoimmune and/or autoinflammatory diseases; undergoing chemotherapy and/or patients that are undergoing immune suppressing treatments that decrease, e.g., wound healing and/or angiogenesis.

The invention provides for the use of nucleotides in concentrations effective to promote wound healing of an animal that is undergoing immunosuppressive therapy. As described in detail herein, a more rapid wound healing reduces recovery time. Concomitant benefits would also include a reduction in medical costs and treatment, time away from work, and the incidence, severity of infection, morbidity and mortality.

The present invention includes compositions and methods for promoting wound healing of an immunosuppressed mammal by preparing a topical composition that comprises one or more compounds selected from the group consisting of nucleotides, nucleosides, nucleobases and mixtures thereof in an amount effective to promote wound healing and contacting a wound of the immunosuppressed mammal with the topical composition. Examples of nucleotides, nucleosides, nucleobases and mixtures thereof include, e.g., RNA, adenine, uracil, inosine, and adenosine in a pharmaceutically acceptable carrier. Other examples of nucleotides, nucleosides, nucleobases and mixtures thereof may even include free pyrimidine bases, nucleosides or nucleotides effective for the promotion of wound healing in a pharmaceutically acceptable carrier. The topical composition may be provided at a dose of about 250 μM to 2.3 M in a pharmaceutically acceptable carrier.

The present invention finds particular uses for the treatment of immunosuppressed mammals, e.g., humans. The wounds that may be treated with the present invention include any trauma to a cell, tissue or organ, e.g., the skin, which may be caused by, e.g., a surgical wound, cutaneous injury, a blunt trauma, etc. When used with a surgical wound, the composition may be administered to the mammal before, during or after a surgery. The composition may be formed into a wound dressing that includes free pyrimidine nucleotides or nucleosides at effective concentration of about 250 μM to 2.3 M. In some particular embodiments, the nucleosides, nucleotides, nucleobases are defined further as pyrimidine bases selected from the group consisting of uracil, thymine and cytosine and/or purine bases such as adenine and guanine.

The present invention also includes a method for the promotion of wound healing in an immunosuppressed subject by preparing a topical composition supplemented with one or more compounds selected from the group consisting of RNA, uracil and adenine as well as corresponding nucleosides and nucleotides (uridine, adenosine, AMP, ADP, ATP, UMP, UDP, UTP) as well as cytosine and guanine, and inosine in an amount effective for promoting wound healing and treating the immunosuppressed subject with the composition such that the wound area has increased concentrations of the compound and wherein the treating is topical. Alternatively, the method may promote wound healing in an immunosuppressed subject by preparing a composition adapted for topical administration in a wound dressing supplemented with one or more compounds selected from the group consisting of RNA, uridine, UMP, UDP, UTP, adenine, uracil, inosine, and adenosine in an amount effective for promoting wound healing; and dressing a wound of the immunosuppressed subject with the wound dressing such that the wound area is at least partially covered from the environment and the wound is contacted to increased concentrations of the compounds.

In another embodiment, the present invention is a method of promoting wound healing in an animal by preparing a wound dressing that includes a pharmaceutically effective amount of one or more compounds selected from the group consisting of nucleotides, nucleosides, nucleobases and mixtures thereof formulated for extended release and placing the wound dressing on a wound, wherein the wound dressing increases the concentration of the compound at or about the wound. A wound dressing foe use with the present invention may be prepared by formulating a pharmaceutically effective amount of one or more compounds selected from the group consisting of nucleotides, nucleosides, nucleobases and mixtures thereof, wherein the compounds are formulated for extended release of at least six hours. Another method of the present invention includes a method for enhancing the rate of wound healing in an animal that includes administering to an animal in need thereof a therapeutically effective concentration of pyrimidine bases, nucleosides or nucleotides or mixtures thereof formulated in a topical pharmacologically acceptable carrier.

Yet another embodiment of the present invention is a method for enhancing the rate of wound healing in an immunosuppressed animal undergoing surgery by administering a composition adapted for topical administration to promote wound healing at a target site that includes an effective amount of pyrimidine bases, nucleosides, nucleotides or mixtures thereof in a pharmacologically acceptable carrier to an immunosuppressed animal prior to surgery. Generally, topical wound dressings are made from a non-allergenic substrate formed into a dressing that include a composition adapted for topical administration to promote angiogenesis at a target site of an immunosuppressed animal having an effective amount of pyrimidine bases, nucleosides or nucleotides or mixtures thereof in a pharmacologically acceptable carrier. The wound dressing may be formed into a suture, a sheet, a compress, a bandage, a band, a prosthesis, a fiber, a woven fiber, a bead, a strip and combinations thereof. For topical use the wound dressing will often be self-adhesive and may include, e.g., a moisture-absorbent material and/or an elastomeric backing.

The present invention may also include a biocompatible and even biodegradable implant that includes a non-allergenic substrate, wherein the implant is formed into a suture, a sheet, a compress, a bandage, a band, a prosthesis, a fiber, a woven fiber, a bead, a strip, clasp, prosthesis, catheter, screw, bone plate, pin, a bandage or combinations thereof and includes a composition adapted for administration to promote wound healing at a target site of an immunosuppressed animal comprising an effective amount of pyrimidine bases, nucleosides or nucleotides or mixtures thereof in a pharmacologically acceptable carrier.

The present invention contemplates a therapeutic agent for the promotion of wound healing. In one embodiment, the therapeutic agent has a therapeutically effective concentration of nucleotides (i.e., effective to promote wound healing) in a pharmacologically acceptable carrier. The nucleotides contained in the “active compound” of the therapeutic agent may comprise RNA, adenine, uridine, any like compounds or a combination thereof. Examples of nucleotides, nucleosides, etc., for use with the present invention may be found in, e.g., U.S. Pat. No. 6,342,484, relevant portions incorporated herein by reference. The nucleotide component may be, e.g., RNA, adenine, uridine, inosine or a mixture thereof. While almost any level of nucleotide administration is expected to be of benefit in the topical wound healing context, it is anticipated that concentrations of about 250 μM to 2.3 M will be particularly useful. These concentrations are for purines and pyrimidines in the form of nucleotides in the pure chemical sense, i.e., with a phosphate group.

One advantage of the present invention is that the topical active compounds (i.e., the nucleotides, nucleosides, etc.) of the present invention may also be administered as a dietary supplement in any convenient manner, such as by the oral, intravenous, intramuscular or subcutaneous routes. The combined dosage regimen (topical and dietary) may be adjusted to provide the optimum therapeutic response. For example, one or more topical doses may be administered daily in conjunction with one or more dietary doses depending on the exigencies of the therapeutic situation. The nucleotides may be administered topically and/or orally alone and/or with an inert diluent, e.g., a carrier adapted for topical delivery. The percentage of the compositions and preparations may, of course, be varied according to the specifics of a therapeutic situation. The amount of active compounds in such therapeutically useful compositions should be such that a suitable dosage will be obtained when a compositions is administered in a suitable way.

Sterile topical solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle that includes, e.g., a basic dispersion medium and the required other ingredients from those enumerated above. In the case of sterile powders for topical administration, these may be prepared as sterile topical solutions. Methods of preparation for the sterile powders may include, e.g., vacuum-drying, spray-freezing, vacuum-heating and freeze-drying techniques that yield a powder of the active ingredient. The composition may even be provided in the form of a kit that contains one or more additional active or inert ingredients and/or carriers (that may also be sterile-filtered) that may be used depending on the circumstances of use and the needs for the location of application. Non-limiting examples of topical applications for the nucleotides include: a serum, saline, gel, jelly, powder, salve, wound dressing, mesh or patch. In the case of wound dressings, these may be in the form of dressings that are, e.g., self-adhesive, water-proof, pads, gauze, biodegradable, biocompatible and combinations thereof.

The present invention also includes methods for promoting wound healing in animals that are undergoing immunosuppressive therapy with, e.g., an effective immunosuppressant that has also been associated with delayed wound healing. The composition and methods include preparing a topical composition of nucleotides effective to promote wound healing and treating the immunosuppressed animal with an effective concentration of the composition. Examples of immunosuppressive agents that may reduce wound healing, may include, e.g., acaricides, actinomycin-D, aldesleukin, aminoglutethimide, amsacrine, anastozole, angiostatin, L-asparaginase, avermectins, azalides, 5-azacytidine, azathioprine, aziridinylbenzoquinone, bafilamycin, bioallenthirn, bleomycin, bicalutamide, brefeldin A, bredinin, bryostatin 1, buserelin, busulfan, carboplatin, carmustine, chivosazol A, chlorambucil, cisplatin, cladribine, colchicinefosfamide, copiamycin, cyclophosphamide, cyproterone, cytarabine, dacarbazine, dactinomycin, daunorubicin, deoxycoformycin, desertomycin, difficidin, diethylstilbestrol, docetaxel, doramectin, doxorubicin, doxycycline, endostatin, epirubicin, eprinmectin, estramustine, etoposide, everolimus, fludarabine, fludrocortisone, 5-fluorodeoxyuridine, 5-fluorouracil, fluoxymesterone, flutamide, geldanamycin, gemcitabine, genistein, grahamimycins, goserelin, hydroxyurea, idarubicin, ifosfamide, ilimaquinone, a-interferon, irinotecan, ivermectin, leucovorin, leuprolide, levamisole, lincomycin, lomustine, mathemycin, mechlorethamine, medroxyprogesterone, megestrol, megovalicins, melphalan, 6-mercaptopurine, mercaptopurine, mesna, methotrexate, minocycline, mithramycin, mitomycin, mitotane, mitoxantrone, moxidectin, nilutamide, nocodazole, okadaic acid, octreotide, oocydin A, oxydifficidin, paclitaxel, pentostatin, plicamycin, porfimer, procarbazine, radicicol, rapamycin, retinoic acid, rhizoxin, sirolimus, staurosporine, streptozocin, sporaviridin, streptogramin, suramin, tamoxifen, tautomycin, teniposide, testolactone, thalidomide, 6-thioguanine, thiotepa, tolytoxin, topotecan, tryphostins, vinblastine, vincristine, vindesine, vinorelbine, virginiamycin, wortmannin, derivatives and combinations thereof.

These methods of the present invention increase the extent of angiogenesis for pre-treating a wound or a wound that may exist in the future, for example, in the case of a scheduled surgery. Thus, the present formulations may be used as part of a pretreatment plan that would provide a heightened level of nucleotides in the immunosuppressed animal or human prior to surgery that will in turn enhance both the healing process and the rate at which the wound is healed. The present invention also contemplates methods of enhancing the rate of wound healing with the administration of a therapeutically effective concentration of nucleotides to an immunosuppressed animal at a wound site.

The present invention contemplates methods encompassing a pretreatment regimen for enhancing the rate of wound healing in an animal that is undergoing immunosuppressive therapy and that may undergo surgery. The topical composition of the present invention may be administrated as a therapeutically effective concentration of nucleotides in a pharmacologically acceptable carrier to an animal at a wound site, e.g., a surgical wound site. In one embodiment, the pretreatment method is expected to involve pretreatment prior to surgery, for example, before, during or following cleaning and/or sterilization of the prospective surgical intervention site. However, benefits of this method can be expected with shorter lengths of pretreatment. Pretreatment methods may include as active compounds, e.g., RNA, adenine, uracil or a mixture thereof as the source of nucleotides. As taught in detail herein, those of skill in the art will understand that other sources of nucleotides will be useful as active compounds in this invention.

The present invention also contemplates a method of preparing a therapeutic agent for the promotion of wound healing comprising placing a wound healing promoting concentration of nucleotides in a pharmacologically acceptable carrier solution at a wound site. The carrier should deliver the nucleotides to an animal in need thereof. The topical preparation may be a liquid, serum, paste, powder, gel, jelly, and the like and may also be provided, alone or in combination with these other dosage forms as a topical wound dressing.

The present invention therefore provides improved therapeutic agents for wound healing, methods for the preparation of these therapeutic agents, and methods for the promotion and enhancement of the rate of wound healing. These compositions and methods are anticipated to provide for a more rapid and complete wound healing in animals. As wound healing is the most catastrophic and costly problem associated with surgery, the advantages of reduced medical complications associated with the healing process and improved quality of wound healing will provide a significant advancement in patient post-surgical clinical management. These and other advantages of the present invention will be further appreciated from the detailed description provided below.

In one important aspect, the present invention may involve preparing a sterile composition comprising, RNA, adenine, uracil, inosine or adenosine in an amount effective to promote wound healing and topically treating the wound of an animal with the composition. Another important embodiment of the present invention for the promotion of wound healing includes the formation of a wound dressing that includes the compositions of the present invention. Topical administration of the nucleotides and possibly protein or amino acids should also be effective.

The pharmaceutical forms suitable for topical use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal and the like. In many cases, the compositions may include isotonic agents, for example, sugars or sodium chloride. Prolonged absorption of the topical compositions can be brought about by the use in the compositions of agents delaying absorption, for example, aluminum monostearate and gelatin.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides preparations and methods of using the preparations for the enhancement of the quality and rate of wound healing in immunosuppressed patients. Methods for preparing the various formulations are also provided for the topical delivery of the compounds of the present invention. As a therapeutic agent, the invention is a concentration of nucleotides together in a pharmaceutical acceptable carrier sufficient to promote wound healing. The therapeutic agents of the invention may be delivered topically to an organism through any of a number of dressings with equal therapeutic efficiency. The methods of the present invention may vary by the wound dressing chosen, the type of wound, the organism treated, the type of immunosuppressive agent and the time-frame of the treatment relative to the time of the wound healing. There are also a variety of methods of preparing the preparations, formulations and/or dressings encompassed within the contemplated scope of the present invention.

As used herein, the terms “active agent,” “pharmacologically active agent” and “drug” are used interchangeably herein to refer to a chemical compound that induces a desired pharmacological, physiological effect, namely wound healing. The terms also encompass pharmaceutically acceptable, pharmacologically active derivatives of those active agents specifically mentioned herein, including, but not limited to, salts, esters, amides, pro-drugs, active metabolites, analogs, and the like. When the terms “active agent,” “pharmacologically active agent” and “drug” are used, then, it is to be understood that applicant intends to include the active agent per se as well as pharmaceutically acceptable, pharmacologically active salts, esters, amides, pro-drugs, metabolites, analogs, etc.

As used herein, the terms “contact”, “contacted”, and “contacting”, are used to describe the process by which a pharmacological agent, e.g., any of the compositions disclosed in the present invention, comes in direct juxtaposition with the target cell(s), tissue or wound site.

As used herein, the term “sustained release” refers to a drug formulation that provides for gradual release of a drug over an extended period of time, and that preferably, although not necessarily, results in substantially constant drug levels at the desired site over an extended period of time.

As used herein, the terms “effective amount” or “therapeutically effective amount” refer to the amount of an agent in a sufficient amount of the agent to provide the desired therapeutic effect. Furthermore, an “effective angiogenesis-inhibiting amount” of an agent is a sufficient amount of the agent to at least partially inhibit angiogenesis. Of course, undesirable effects, e.g., side effects, are sometimes manifested along with the desired therapeutic effect; hence a practitioner balances the potential benefits against the potential risks in determining what is an appropriate “effective amount” at the site or target of delivery of the active agent. The exact amount required will vary from subject to subject, depending on the species, age, and general condition of the subject, mode of administration, and the like. Thus, it is not possible to specify an exact “effective amount.” However, an appropriate “effective amount” or “effective wound healing amount” in any individual case may be determined by one of ordinary skill in the art using only routine experimentation.

As used herein, the term “pharmaceutically acceptable” carrier refers to a pharmaceutical vehicle comprised of a material that is not biologically or otherwise undesirable, i.e., the material may be administered to an individual along with the selected active agent without causing any undesirable biological effects or interacting in a deleterious manner with any of the other components of the pharmaceutical formulation in which it is contained. Carriers may include excipients and other additives such as diluents, detergents, coloring agents, wetting or emulsifying agents, pH buffering agents, preservatives, and the like.

Of course, the nucleotide active compounds of the present invention may be administered topically by any of the other numerous techniques known to those of skill in the art as taught by, e.g., Remington's Pharmaceutical Science 18th Edition, (1990), which is specifically incorporated herein in pertinent part for this purpose. Supplementary active ingredients can also be incorporated into the compositions, e.g., analgesics, antibiotics and the like.

As used herein, the term a “pharmacologically acceptable” salt, ester, amide, prodrug, or derivative of a compound refers to a salt, ester, amide, prodrug, or derivative that is not biologically or otherwise undesirable.

As used herein, the term “substrate,” or “solid support” and “wound dressing” refer broadly to any substrate when prepared for, and applied to, a wound for protection, absorbance, drainage, etc. The present invention may include any one of the numerous types of substrates and/or backings that are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (non-woven composites of fibers from calcium alginate), and cellophane (cellulose with a plasticizer). The shape and size of a wound may be determined and the wound dressing customized for the exact site based on the measurements provided for the wound. As wound sites can vary in terms of mechanical strength, thickness, sensitivity, etc., the substrate can be molded to specifically address the mechanical and/or other needs of the site. For example, the thickness of the substrate may be minimized for locations that are highly enervated, e.g., the finger tips. Other wound sites, e.g., fingers, ankles, knees, elbows and the like, may be exposed to higher mechanical stress and require multiple layers of the substrate.

As used herein, the terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual and treatment of a surgical, blunt or other trauma of the skin by increasing healing at a wound site. Thus, for example, the present method of “treating” a patient in need of wound healing therapy encompasses both prevention of a condition, disease, or disorder that is responsive to anti-rejection or immunosuppressive therapy (e.g., transplant rejection) and the treatment of a wound site that is in need of healing.

As used herein, the term “patient” refers to a mammalian, e.g., a human, which can benefit from the pharmaceutical formulations and methods of the present invention. There is no limitation on the type of mammal that could benefit from the presently described pharmaceutical formulations and methods.

As used herein, the term “wound healing” refers to trauma and injury of a cell, tissue or organ. Such sudden and external trauma injury requires intact and able host defense mechanisms.

### EXAMPLE

### Preparation of Nucleotide-Containing Therapeutic Composition for Humans

The present example is provided to detail the proposed preparation of a nucleotide-enriched composition suitable for topical administration to humans. These compositions can contain any combination of nucleotides. However, the inventors have made preliminary observations suggesting that combinations that include, e.g., purines and pyrimidines or simply pyrimidines alone.

For example, a nucleotide-enriched topical serum may contain about 250 μM to 2.3 M RNA, dissolved in water or another suitable liquid. To make such a preparation, 2.5 g of RNA (from yeast or another source) are mixed with a diluent, e.g., water or saline. It may also be necessary to add additional carriers to place the liquid in a suitable form for providing the patient with a topical composition that does not irritate the skin, e.g., providing the RNA in conjunction with a gel, e.g., aloe vera gel, glycerol, glycerin, KY-like jellies, vitamin E or other carriers known to be compatible with topical applications.

### EXAMPLE

### Methods for Promoting/Enhancing the Rate of Wound Healing in Humans

The present example is provided to outline proposed methods for the nucleotide regimens of the invention may be used in the treatment of humans for the promotion/enhancement of wound healing. Examples of use of RNA or nucleotide sources as a topical substrate for enhancing wound healing would include some of the following clinical uses. Note that in the following example, RNA can be substituted for similar levels of purines and/or pyrimidines.

RNA or nucleosides can be administered topically via salve, an ointment, an impregnated dressing, a sustained release patch, or as a powder. In this application the substrate can be applied directly to the wound to induce enhanced healing. The dose will range from 0.250 mM to 2.5 M.

### EXAMPLE

### Topical Applications for Wound Healing in Mice

Balb/c mice were divided into three dietary groups (F=normal mouse chow; NF=nucleotide free mouse chow; and NFR=nucleotide free mouse chow supplemented with yeast RNA). Each group was fed before initiation of wounds. After 4 weeks of feeding, a 4 mm punch wound was created on the dorsal surface of each mouse, using a 4 mm biopsy punch. The wounds were coated with four different topical agents: (1) saline; (2) uracil (100 M concentration); (3) saline+CCM; and (4) Uracil+CCM (CCM=crosslinked collagen mesh). CCM (“Collagen Permeable Membrane”) from Accurate Chemical & Scientific Corporation, Catalog No. YIC 152299. Four mm size circles were soaked with appropriate nucleotide or saline solution (4 mm punch wounds) and used to dress the wounds.

Twelve groups were formed from these combinations (n=3). Mice were continued on their respective diets. Wounds were observed visually and photographed at four different times during the period of two weeks. These studies were repeated using a 100 μM concentration of inosine in place of uracil as the topical agent. The results yielded no significant differences from that which was seen with the uracil.

### EXAMPLE

### Wound Healing and Immunosuppression

Sirolimus and everolimus are two macrolide immunosuppressants increasingly used in organ and cell transplantation because they effectively suppress allograft rejection responses while not impairing renal function, unlike calcineurin inhibitors. The mechanism of action of these agents appears to be blocking of signal transduction from cell surface membrane receptors to the nucleus.

A powerful side effect observed for immunosuppressive agents include decreased wound healing, presumably secondary to the observed suppression of vascular endothelial growth factor (VEGF), a critical protein in wound angiogenesis. For example, Sirolimus also decreases response to platelet derived growth factor (PDGF), a protein which also enhances angiogenesis.

Sirolimus is an effective immunosuppressant that has been associated with delayed wound healing. On a full thickness skin murine model of immunosuppression and decreased wound healing, the Sirolimus dose that delayed wound healing was first determined. Next, an effective dose of topical administration of mixed nucleotides was determined that demonstrated enhanced wound healing despite Sirolimus treatment.

Methods: For 2 weeks Balb/c mice were gavage-fed saline, 4 mg/kg, 8 mg/kg or 12 mg/kg Sirolimus (10 per group). After drug loading, an 8 mm in diameter full thickness dorsal skin punch wound was made in each mouse. Every second day digital pictures of the wound were taken and analyzed. After determining the effective Sirolimus dose that causes decreased wound healing and immunosuppression, 40 Balb/c mice were gavaged with 8 mg/kg of Sirolimus every other day. After establishing the punch wounds, the mono and dinucleotides were applied topically daily. The topical treatment was applied using the following formulations (10 mice per group): a mixture of mono and dinucleotides derived from RNA in 0.025%, 0.25% and 2.5% concentrations and one group with KY jelly as control. Every second day a digital picture was taken and wound size measured. P2Y2 receptor expression was qualitatively assessed using immunohistology and receptor specific antibodies. T-test was used to compare the groups.

Results. It was found that at 4 mg/kg Sirolimus dose showed no effect on wound healing. An 8 mg/kg and 12 mg/kg doses on day 4 and after delayed wound healing in a significant manner (p=0.01). A significant difference was found during the whole healing period.

Following this dose response test, an effective dose of 8 mg/kg was established for Sirolimus. It was found that at: 0.25% and 2.5% mixed RNA, the wound size of animals treated topically decreased faster after day 2 and this decrease turned to be significant on day 6 (p=0.02 and p=0.025). This statistically significant enhanced wound healing was observed through day 10. On day 14, all wounds were healed.

These studies demonstrate that mixed RNA applied topically was capable of overcoming the effect of an immunosuppressive agent on wound healing. It was found that Sirolimus impaired wound healing in this well-known model system was overcome, when mixed RNA was applied topically. Furthermore, it was found that mixed RNA accelerates wound healing in these immunosuppressive drug-treated animals. The qualitative assessment of the P2Y2 receptors suggests that binding to these receptors may be the mechanism by which the positive influence of the nucleotides applied topically are mediated. This effective treatment offers an opportunity to mitigate a complication of Sirolimus therapy.

TABLE 1
Wound size percent during the follow-up period.

| | | Day 0 | Day 2 | Day 4 | Day 6 | Day 8 | Day 10 | Day 12 | Day 14 |
|---|---|---|---|---|---|---|---|---|---|
| KY Jelly | mean | 100 | 98.19 | 87.17 | 77.04 | 67.99 | 62.57 | 18.62 | 7.85 |
| | SD | 0 | 3.47 | 9.29 | 12.85 | 20.82 | 22.26 | 12.87 | 8.51 |
| 0.025% mixed | mean | 100 | 96.77 | 84.93 | 74.53 | 66.64 | 52.57 | 16.36 | 6.35 |
| nucleotide | SD | 0 | 5.19 | 9.71 | 11.21 | 11.18 | 21.06 | 8.73 | 5.95 |
| 0.25% mixed | mean | 100 | 90.35 | 72.01 | 57.14 | 43.37 | 29.87 | 10.99 | 4.88 |
| nucleotide | SD | 0 | 14.01 | 21.34 | 19.28 | 21.77 | 21.63 | 8.68 | 6.67 |
| 2.5% mixed | mean | 100 | 93.44 | 81.56 | 59.94 | 52.94 | 41.68 | 12.33 | 6.41 |
| nucleotide | SD | 0 | 10.4 | 17.22 | 15.15 | 20.04 | 18.57 | 4.9 | 4.98 |

TABLE 2
P values as the mixed nucleotide topical treated groups compared to the
KY jelly group.

| Mixed RNA | Day 0 | Day 2 | Day 4 | Day 6 | Day 8 | Day 10 | Day 12 | Day 14 |
|---|---|---|---|---|---|---|---|---|
| 0.025% | n/a | 0.27 | 0.32 | 0.34 | 0.44 | 0.19 | 0.35 | 0.34 |
| 0.25% | n/a | 0.08 | 0.05 | 0.01 | 0.02 | 0.006 | 0.09 | 0.23 |
| 2.50% | n/a | 0.08 | 0.23 | 0.02 | 0.09 | 0.04 | 0.12 | 0.35 |

Therefore, RNA derived nucleotides, nucleosides or nucleobases when applied topically to wounds in mice treated with an immunosuppressive dose of sirolimus (8 mg/kg/every other day) significantly enhance wound healing in mice despite sirolimus therapy. The effective dose ranges from 250 μM to 2.3 M as a topical treatment of nucleosides, nucleotides, or nucleobases. At least one mechanism of this effect of the topical compounds appears to be via enhancing angiogenesis in the wound.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Caspe, S. (1981) U.S. Pat. No. 4,308,257.

Chou, et al. (1984) U.S. Pat. No. 4,486,439.

Gil, et al. (1991) “Method for stimulation or repair and regeneration of intestinal gut cells in infants and enhancing the immune response of T-cells,” U.S. Pat. No. 4,994,442.

Gil, et al. (1991) “Infant formulas and nutrition products enriched with nucleosides and/or nucleotides and processes for their preparation,” U.S. Pat. No. 5,066,500.

Studt, et al. (1984) “Treatment of coccidiosis,” U.S. Pat. No. 4,486,439.

Belardinelli, et al., The Candiac Effects of Adenosine, Progress in Cardiovascular Diseases, XXXII:73-97 (1989).

DeLucchi, et al. (1991) “Effects of dietary nucleotides on the fatty acid composition of erythrocyte membrane lipids in term infants,” J. Pediatr. Gastroenterol. Nutr., 6(4):568-574.

Faist, et al. (1991) “Nutrition and tumor diseases,” In: Surgery Nutrition and the Immune System, P. Schauder (Ed.)

Fanslow, et al. (1988) “Effects of nucleotide restriction and supplementation on resistance to experimental murine candidiasis.” J Parent Ent Nutr 12:49-52.

Gauri (1983) “Combined preparation with synergistic effect (translation from German,” K.K. EP No. 85,084.

Goodson W. H. and Hunt T. K. (1982) “Development of a new miniature method for the study of wound healing in human subjects,” J. Surg. Res. 33:394-401.

Hunt, Goodson, and Scheuenstuhl, “A strategy for human studies: Thoughts on models” Wound Healing: Ed. H. Janssen, R. Rooman and J. I. S. Robertson, 1991 Wrightson Biomedical Publishing Ltd.

Kulkarni, et al. (1992) “Tmmunohemopoietic effects of dietary nucleotide restriction in mice,” Transplantation 53(2):467-472.

Kulkarni, et al. (1988) “Expression of immune cell surface markers in vivo and immune competence in mice by dietary nucleotides,” Transplantation Proc. 21(1):121-124.

Kulkarni, et al. (1986) Influence of dietary nucleotide restriction on bacterial sepsis and phagocytic cell function in mice. Archives of Surgery 121:169-172.

Kulkarni, et al. (1987) “Modulation of delayed hypersensitivity in mice by dietary nucleotide restriction,” Transplantation 44(6):847-49.

Mauch, Adelman-Grill, Hatamochi, and Krieg (1989) “Collagenase gene expression in fibroblasts is regulated by a three-dimensional contact with collagen” FEBS Letters 250:301-305.

Nelson T. S. and Anders C. J. (1966) “Dynamic aspects of small intestinal rupture with special consideration of anastomotic strength,” Arch. of Surg. 93:309-314.

Peterson J. M. et al. (1987) “Significance of T-lymphocytes in wound healing,” Surgery 102(2):300-305.

Pizzini, et al. (1990) “Dietary nucleotides reverse malnutrition and starvation-induced immunosuppression,” Arch. Surg. 125(1):86-89.

Rudolph F. B., et al. (1990) “Role of RNA as a dietary source of pyrimidines and purines in immune function,” Nutrition 6:45-51.

Van Buren, et al. (1983) “The influence of dietary nucleotides on cell mediated immunity,” Transplantation 36:350-352.

Van Buren, et al. (1985) “Dietary nucleotides: A requirement for helper/suppressor lymphocytes,” Transplantation, 40(6):694-697.

Woessner J. F. Jr. (1961) “The determination of hydroxyproline in tissue and protein samples containing small proportions of this amino acid,” Arch. Biochem. Biophys. 93:440-447.

What is claimed is:

1. A method for promoting wound healing of an immunosuppressed mammal comprising:
preparing a topical composition that comprises one or more compounds selected from the group consisting of nucleotides, nucleosides, nucleobases and mixtures thereof in an amount effective to promote wound healing; and
contacting a wound of the immunosuppressed mammal with the topical composition.

2. The method of claim 1, wherein the nucleotides, nucleosides, nucleobases and mixtures thereof are further defined as comprising RNA, adenine, uracil, inosine, and adenosine in a pharmaceutically acceptable carrier.

3. The method of claim 1, wherein the nucleotides, nucleosides, nucleobases and mixtures thereof are further defined as comprising free pyrimidine bases, nucleosides or nucleotides effective for the promotion of wound healing in a pharmaceutically acceptable carrier.

4. The method of claim 1, wherein the topical composition comprises a dose of about 250 μM to 2.3 M in a pharmaceutically acceptable carrier.

5. The method of claim 1, wherein the immunosuppressed mammal is a human.

6. The method of claim 1, wherein the wound is a surgical wound.

7. The method of claim 1, wherein the wound is a surgical wound and the composition is administered to the mammal before, during or after a surgery.

8. The method of claim 1, wherein the topical composition comprises free pyrimidine nucleotides or nucleosides.

9. The method of claim 1, wherein the immunosuppressed mammal is treated with a composition of nucleosides, nucleotides, nucleobases and combinations thereof at an effective concentration of about 250 μM to 2.3 M.

10. The method of claim 1, wherein the animal is treated topically with nucleosides, nucleotides, nucleobases and combinations thereof provided at about 250 μM to 2.3 M.

11. The method of claim 1, wherein the nucleosides, nucleotides, nucleobases are defined further as pyrimidine bases selected from the group consisting of uracil, thymine and cytosine.

12. The method of claim 1, wherein the mammal is under treated with a pharmaceutical immunosuppressive agent.

13. The method of claim 1, wherein the mammal is under treated with a pharmaceutical immunosuppressive agent selected from the group consisting of acaricides, actinomycin-D, aldesleukin, aminoglutethimide, amsacrine, anastozole, angiostatin, L-asparaginase, avermectins, azalides, 5-azacytidine, aziridinylbenzoquinone, bafilamycin, bioallenthim, bleomycin, bicalutamide, brefeldin A, bredinin, bryostatin 1, buserelin, busulfan, carboplatin, carmustine, chivosazol A, chlorambucil, cisplatin, cladribine, colchicinefosfamide, copiamycin, cyclophosphamide, cyproterone, cytarabine, dacarbazine, dactinomycin, daunorubicin, deoxycoformycin, desertomycin, difficidin, diethylstilbestrol, docetaxel, doramectin, doxorubicin, doxycycline, endostatin, epirubicin, eprinmectin, estramustine, etoposide, fludarabine, fludrocortisone, 5-fluorodeoxyuridine, 5-fluorouracil, fluoxymesterone, flutamide, geldanamycin, gemcitabine, genistein, grahamimycins, goserelin, hydroxyurea, idarubicin, ifosfamide, ilimaquinone, α-interferon, irinotecan, ivermectin, leucovorin, leuprolide, levamisole, lincomycin, lomustine, mathemycin, mechlorethamine, medroxyprogesterone, megestrol, megovalicins, melphalan, mercaptopurine, mesna, methotrexate, minocycline, mithramycin, mitomycin, mitotane, mitoxantrone, moxidectin, nilutamide, nocodazole, okadaic acid, octreotide, oocydin A, oxydifficidin, paclitaxel, pentostatin, plicamycin, porfimer, procarbazine, radicicol, rapamycin, retinoic acid, rhizoxin, sirolimus, staurosporine, streptozocin, sporaviridin, streptogramin, suramin, tamoxifen, tautomycin, teniposide, testolactone, 6-thioguanine, thiotepa, tolytoxin, topotecan, tryphostins, vinblastine, vincristine, vindesine, vinorelbine, virginiamycin, wortmannin, derivatives and combinations thereof.

14. A method for the promotion of wound healing in an immunosuppressed subject comprising:
preparing a topical composition supplemented with one or more compounds selected from the group consisting of RNA, uracil and inosine in an amount effective for promoting wound healing; and
treating the immunosuppressed subject with the composition such that the wound area has increased concentrations of the compound and wherein the treating is topical.

15. A method for the promotion wound healing in an immunosuppressed subject comprising:
preparing a composition adapted for topical administration in a wound dressing supplemented with one or more compounds selected from the group consisting of RNA, adenine, uracil, inosine, and adenosine in an amount effective for promoting wound healing; and
dressing a wound of the immunosuppressed subject with the wound dressing such that the wound area is at least partially covered from the environment and the wound is contacted to increased concentrations of the compounds.

16. A method of promoting wound healing in an animal comprising:
preparing a wound dressing comprising a pharmaceutically effective amount of one or more compounds selected from the group consisting of nucleotides, nucleosides, nucleobases and mixtures thereof formulated for extended release; and
placing the wound dressing on a wound, wherein the wound dressing increases the concentration of the compound at or about the wound.

17. A wound dressing prepared by formulating a pharmaceutically effective amount of one or more compounds selected from the group consisting of nucleotides, nucleosides, nucleobases and mixtures thereof, wherein the compounds are formulated for extended release of at least six hours.

18. A method for enhancing the rate of wound healing in an animal comprising administering to an animal in need thereof a therapeutically effective concentration of pyrimidine bases, nucleosides or nucleotides or mixtures thereof formulated in a topical pharmacologically acceptable carrier.

19. A method for enhancing the rate of wound healing in an immunosuppressed animal undergoing surgery comprising:
administering a composition adapted for topical administration to promote wound healing at a target site comprising an effective amount of pyrimidine bases, nucleosides, nucleotides or mixtures thereof in a pharmacologically acceptable carrier to an immunosuppressed animal prior to surgery.

20. A wound dressing comprising a non-allergenic substrate formed into a dressing comprising a composition adapted for topical administration to promote angiogenesis at a target site of an immunosuppressed animal comprising an effective amount of pyrimidine bases, nucleosides or nucleotides or mixtures thereof in a pharmacologically acceptable carrier.

21. The wound dressing of claim 20, wherein the wound dressing is formed into a suture, a sheet, a compress, a bandage, a band, a prosthesis, a fiber, a woven fiber, a bead, a strip and combinations thereof.

22. The wound dressing of claim 20, wherein the wound dressing is self-adhesive.

22. The wound dressing of claim 20, wherein the wound dressing comprises a moisture-absorbent material and an elastomeric backing.

24. A biocompatible implant comprising:
a non-allergenic substrate, wherein the implant is formed into a suture, a sheet, a compress, a bandage, a band, a prosthesis, a fiber, a woven fiber, a bead, a strip, clasp, prosthesis, catheter, screw, bone plate, pin, a bandage or combinations thereof and comprises a composition adapted for administration to promote wound healing at a target site of an immunosuppressed animal comprising an effective amount of pyrimidine bases, nucleosides or nucleotides or mixtures thereof in a pharmacologically acceptable carrier.

25. A method of maintaining a wound healing environment during immunosuppressive therapy comprising applying a topical formulation at the wound site comprising nucleotides, nucleosides, nucleobases and mixtures thereof in an amount effective to overcome the effect of the immunosuppressive therapy on wound healing.
